(19) United States
(12) Patent Application Publication
(10) Pub. No.: US 2022/0160484 A1
(43) Pub. Date: May 26, 2022

(54) MOUTHPIECE-TYPE ELECTRIC TOOTHBRUSH AND CLEANING DEVICE THEREFOR

(71) Applicant: Mu Se KIM, Gwangju (KR)

(72) Inventor: Mu Se KIM, Gwangju (KR)

(21) Appl. No.: 17/439,702

(22) PCT Filed: Nov. 22, 2019
(86) PCT No.: PCT/KR2019/016203

§ 371 Date: Sep. 15, 2021

(30) Foreign Application Priority Data
Mar. 22, 2019 (KR) 10-2019-0033184
Nov. 18, 2019 (KR) 10-2019-0147870

(51) Int. Cl. A61C 17/20; A61C 17/22; A46B 9/04; A46B 11/00; A46B 17/06; A61L 2/26

(52) U.S. Cl. CPC A61C 17/20; A61C 17/228; A46B 9/045; A46B 11/002; A61L 2202/11; A46B 17/065; A61L 2/26; A61C 2201/00; A61C 17/224

(57) ABSTRACT

Proposed is a mouthpiece-type electric toothbrush. The mouthpiece-type electric toothbrush includes a toothbrush body a first tooth insertion groove in which the upper teeth of a user are inserted and a second tooth insertion groove in which the lower teeth are inserted. The toothbrush body has a plurality of bristles having different lengths and formed in the first tooth insertion groove and the second tooth insertion groove in accordance with a tooth mold patterned after a row of teeth of the user.

### TECHNICAL FIELD

The present disclosure relates to a mouthpiece-type electric toothbrush and a cleaning device therefor and, more particularly, to a mouthpiece-type electric toothbrush which can clean teeth in a custom-fit type for users having different rows of teeth.

### BACKGROUND ART

In general, since most electric toothbrushes mechanically rotate or vibrate bristles using an electric motor, there is a problem that they can clean the surface of teeth, but cannot clean the small gaps between the gums and the teeth and the soft gums are damaged by a strong brushing effect. Damage to the gums causes injuries in the mouth, which causes inflammation.

Further, a high-speed toothbrush that vibrates an electronic magnet 37,000 or 15,000 times per second by increasing the driving frequency of the electronic magnet up to an ultrasonic level has been commercialized, but this toothbrush cannot solve the defects of vibration toothbrushes because the amplitude is large and strong. Further, it is difficult to expect the sterilizing and cleaning effect by a strong cavitation effect from the vibration of 15,000 times per second.

In order to solve these problems, an ultrasonic toothbrush that uses a piezoelectric ultrasonic method is recently recognized as having high usability in the medical field. However, according to the toothbrushes using a piezoelectric ultrasonic method, a piezoelectric member that generates ultrasonic vibration is attached to the plastic where the bristles are mounted, and a vibration mode of 1.6 MHz is used.

According to these toothbrushes of the related art, the ultrasonic waves generated by the piezoelectric member are attenuated by the plastic of the toothbrushes, so ultrasonic vibration is hardly generated. Further, the toothbrushes of the related art use only the effect by ultrasonic acoustic transfer and employ a vibration motor type for mechanical vibration, so it is difficult to obtain effectively ultrasonic vibration energy and a cavitation effect using the piezoelectric member. Accordingly, it is impossible to solve the defects of toothbrushes using the vibration motor type in the related art.

Therefore, there is a need for a more effective ultrasonic toothbrush that can generate both ultrasonic vibration and the cavitation effect.

An “Ultrasonic washer for teeth” has been disclosed in Korean Utility Model No. 20-0440596 and a “Mouthpiece type automatic toothbrush” has been disclosed in Korean Utility Model No. 20-0470142.

Further, a “Mouthpiece type dental automatic cleaning apparatus” has been disclosed in Korean Patent No. 10-1781631. The dental automatic cleaning apparatus includes: a body formed in a U-shape to correspond to the oral structure of users, and having upper grooves formed to insert upper teeth on the upper surface and lower grooves formed to insert lower teeth on the lower surface; and a handle disposed on the front of the body. The body has an upper tray having an upper opening and an upper bristle member attached to an upper shock-absorbing member, which is spaced a predetermined distance apart from the bottom of the upper tray, and having several bristles.

Further, an “Ultrasonic washer for teeth” has been disclosed in Korean Patent Application Publication No. 10-2011-0111908. The ultrasonic washer for teeth includes: a mouthpiece type ultrasonic wave generator that has an H-shaped cross-section with an upper tooth insertion groove and a lower tooth insertion groove and is formed in a U-shape; and a control box that controls the operation of the mouthpiece type ultrasonic wave generator.

A system for axial motion of bristles of a tooth-cleaning mouthpiece has been disclosed in Japanese Patent No. 5698148 and an acoustic vibration tooth cleaning device has been disclosed in Korean Patent No. 1661108.

The acoustic vibration tooth cleaning device includes: a housing that has tooth insertion grooves for inserting the teeth of a user and has bristles in the tooth insertion grooves; gap adjustment member grooves formed in the housing; a pair of gap adjustment members that is inserted in the gap adjustment member grooves, partially protrudes from a side of the housing such that the protruding portions cross each other, and has a hinge member; an operation member that adjusts the gap of the gap adjustment members; and a handle in which the operation member is inserted.

The tooth washer having this configuration is not fully inserted in a mouth and the handle protrudes between lips, so there is a problem that a cleaning fluid leaks between lips. Further, the tooth washer generally cleans teeth with the teeth inserted in the tooth insertion grooves of the housing, so the tongue is not cleaned.

Further, there is a problem that since back teeth are cleaned only on both sides by the bristles having the tooth insertion grooves, the inner surfaces of the back teeth are not cleaned. Further bubbles are generated when ultrasonic waves are generated with the cleaning fluid and the salvia in the tooth insertion grooves, and the generated bubbles overflow the tops of the tooth insertion grooves, but lips cannot be completely closed due to vibration of ultrasonic waves and the handle. Accordingly, there is a problem that the cleaning fluid and salvia fly around the lips, thereby contaminating the parts around the lips.

Further, there is a problem in the related art that since bristles are uniformly formed, the bristles cannot reach all teeth because people have different tooth shapes and oral structure. Further, cleaning is important to remove foreign matters between teeth, but the bristles cannot come into between teeth and remove foreign matters, so cleaning is not performed well.

### DISCLOSURE

### Technical Problem

An objective of the present disclosure is to provide a mouthpiece-type electric toothbrush which can solve the problem that a cleaning fluid and bubbles flow down or fly out of a mouth when cleaning teeth, which can clean a tongue, which can guide a cleaning fluid and bubbles generated from tooth insertion grooves formed at a toothbrush body into the mouth, an which can improve the efficiency of cleaning teeth by cleaning the sides of the rearmost back teeth.

Another objective of the present disclosure is to provide a mouthpiece-type electric toothbrush which has a high cleaning efficiency because it is formed to fit the rows of teeth of users.

The objectives of the present disclosure are not limited to those described above and other objectives may be made apparent to those skilled in the art from claims.

### Technical Solution

In order to achieve the objectives of the present disclosure, a mouthpiece-type electric toothbrush according to an embodiment of the present disclosure includes a toothbrush body having a first tooth insertion groove in which the upper teeth of a user are inserted and a second tooth insertion groove in which the lower teeth of a user are inserted, in which the toothbrush body has a plurality of bristles having different lengths and formed in the first tooth insertion groove and the second tooth insertion groove in accordance with a tooth mold patterned after a row of teeth of a user.

The toothbrush body may be formed by inserting a thermoplastic material to fit the row of teeth of a user at a predetermined temperature.

A mouthpiece-type electric toothbrush includes: a toothbrush body having a first tooth insertion groove in which the upper teeth of a user are inserted and a second tooth insertion groove in which the lower teeth of the user are inserted; an actuator body having a ultrasonic vibration generator mounted on the toothbrush body and transmitting vibration to the first tooth insertion groove and the second tooth insertion groove; and a body cradle on which the actuator body is placed and that is configured to wash and/or dry the first tooth insertion groove and the second tooth insertion groove.

The body cradle may have a body seat on which the actuator body is seated, and a charging module configured to charge the actuator body may be disposed at the body seat.

The body cradle may have recessed grooves that are respectively formed at both sides with the body seat therebetween and at which the first tooth insertion groove and the second tooth insertion groove are disposed, and a sterilizing module configured to dry and/or sterilize the toothbrush body may be disposed at each of the recessed grooves.

The sterilizing module may be any one of a UV lamp and/or a blowing fan.

The sterilizing modules may be inclined at a predetermined angle toward the first tooth insertion groove and the second tooth insertion groove.

The mouthpiece-type electric toothbrush may further include a cover applied by coating to block UV light that is emitted from the UV lamp.

### Advantageous Effects

According to the mouthpiece-type electric toothbrush of the present disclosure and a cleaning device for the mouthpiece-type electric toothbrush, the following one or more effects can be achieved.

First, according to the mouthpiece-type electric toothbrush of the present disclosure and a cleaning device for the mouthpiece-type electric toothbrush, since teeth are cleaned with the electric toothbrush inserted in a mouth, it is possible to prevent a cleaning fluid for the teeth and bubbles from flowing or flying out of the mouth.

Second, according to the mouthpiece-type electric toothbrush of the present disclosure and a cleaning device for the mouthpiece-type electric toothbrush, since the first and second discharge guide grooves are formed in the second and fourth sides, respectively, the cleaning fluid and bubbles can be smoothly discharged from the first and second tooth insertion grooves and can smoothly flow into the first and second tooth insertion grooves from the oral cavity.

Third, according to the mouthpiece-type electric toothbrush of the present disclosure and a cleaning device for the mouthpiece-type electric toothbrush, since the vibration generated by the first, second, and third ultrasonic vibration motors can be transmitted well to the parts of the toothbrush body by the vibration accelerator disposed in the toothbrush body, the power for cleaning teeth can be increased.

Fourth, according to the mouthpiece-type electric toothbrush of the present disclosure and a cleaning device for the mouthpiece-type electric toothbrush, since a user can freely use both hands while the teeth are cleaned, it is possible to reduce the inconvenience that the user has to brush his/her teeth when he/she is busy, and the disabled and the elderly and weak who have difficulty in moving both hands can easily brush their teeth without help by an assistant.

Fifth, according to the mouthpiece-type electric toothbrush of the present disclosure and a cleaning device for the mouthpiece-type electric toothbrush, it is possible to expect the effect of not only cleaning teeth, but massaging the gums and it is possible to remove plaque and tartar using ultrasonic vibration.

Sixth, according to the mouthpiece-type electric toothbrush of the present disclosure and a cleaning device for the mouthpiece-type electric toothbrush, a delay time of several seconds is provided until the actuator body is operated after liquid toothpaste is applied to the toothbrush body and an operation switch is turned on before the toothbrush is put into a mouth, whereby it is possible to prevent the toothpaste from flying outside the mouth.

Seventh, according to the mouthpiece-type electric toothbrush of the present disclosure and a cleaning device for the mouthpiece-type electric toothbrush, the secondary battery for driving the ultrasonic vibration generator of the actuator body can be wirelessly charged and the actuator body has a shield for blocking electromagnetic waves, so it is possible to fundamentally prevent problems due to electromagnetic waves in a mouth.

Eighth, according to the mouthpiece-type electric toothbrush of the present disclosure and a cleaning device for the mouthpiece-type electric toothbrush, there is an effect that since even though a user has a set of non-uniform teeth, the bristles can remove foreign matters on the teeth and between the teeth because the bristles are formed to fit to the shapes of the teeth and the oral structure of the user.

Ninth, according to the mouthpiece-type electric toothbrush of the present disclosure and a cleaning device for the mouthpiece-type electric toothbrush, there is an effect that the mouthpiece-type electric toothbrush is pasteurized and sterilized for a predetermined time through the body cradle on which the mouthpiece-type electric toothbrush is placed, so the mouthpiece-type electric toothbrush can be always provided in a clean state.

Tenth, according to the mouthpiece-type electric toothbrush of the present disclosure and a cleaning device for the mouthpiece-type electric toothbrush, there is an effect that when the mouthpiece-type electric toothbrush is placed on the body cradle, it can be wirelessly charged, so there is an effect that convenience is improved.

The effects of the present disclosure are not limited to those described above and other effects not stated herein may be made apparent to those skilled in the art from claims.

### DESCRIPTION OF DRAWINGS

FIG. 1 is a perspective view of a mouthpiece-type electric toothbrush according to the present disclosure;

FIG. 2 is a cross-sectional view of the mouthpiece-type electric toothbrush shown in FIG. 1;

FIG. 3 is an exploded perspective view showing a toothbrush body and an actuator body of the mouthpiece-type electric toothbrush according to the present disclosure;

FIG. 4 is a perspective view showing a portion of the mouthpiece-type electric toothbrush;

FIG. 5 is a plan view showing the mouthpiece-type electric toothbrush that has been formed to fit a tooth mold of teeth;

FIG. 6 is a cross-sectional view showing a mouthpiece-type electric toothbrush according to an embodiment of the present disclosure; and

FIG. 7 is a projective front view showing the state in which a mouthpiece-type electric toothbrush according to an embodiment of the present disclosure has been mounted on a cleaning device.

### MODE FOR INVENTION

Before the present disclosure is described in detail below, it should be understood that the terms used herein are provided only to describe specific embodiments without limiting the scope of the present disclosure is limited only by claims. All technical terminologies and scientific terminologies used herein have the meanings that are understood by those skilled in the art unless stated otherwise.

The terms ‘comprise’, ‘comprises’, and ‘comprising’ used throughout the specification and claims have a meaning that includes stated objects and steps or sets of objects and steps unless stated otherwise without excluding other certain objects and steps, or a set of objects, or a set of steps.

Several embodiments of the present disclosure may be combined with any other embodiments unless an opposite meaning is clearly stated. In particular, any characteristics stated as being preferable or advantageous may be combined with other characteristics stated as being preferable or advantageous and other characteristics. Embodiments of the present disclosure and corresponding effects are described hereafter with reference to the accompanying drawings.

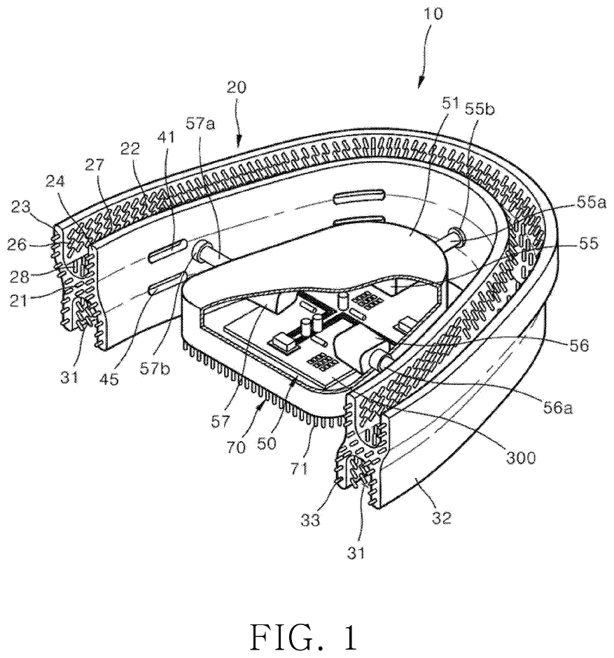

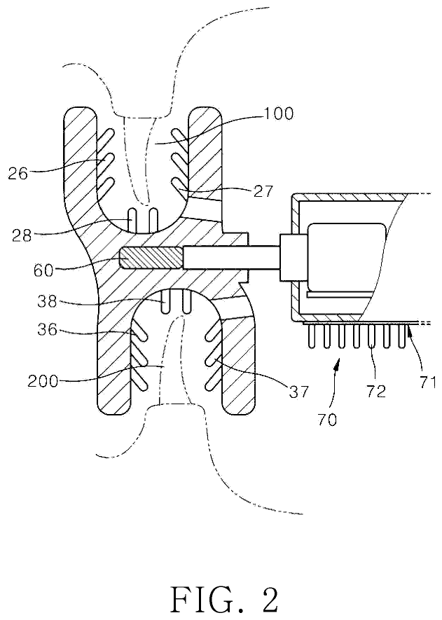

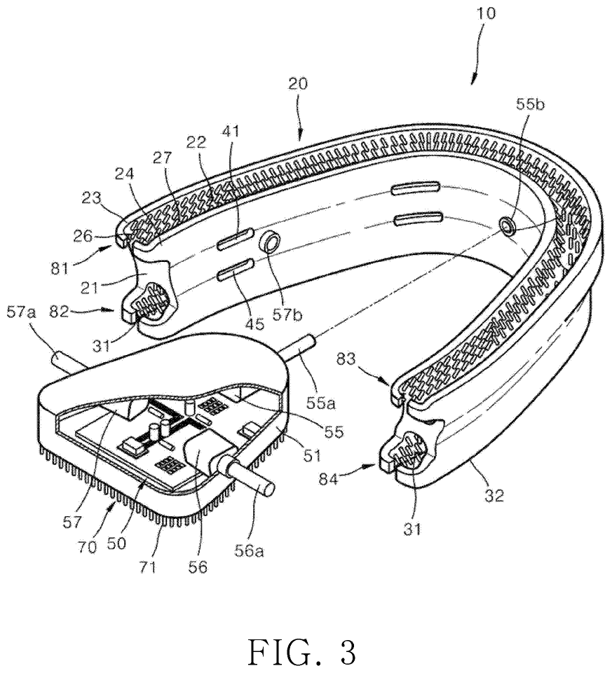

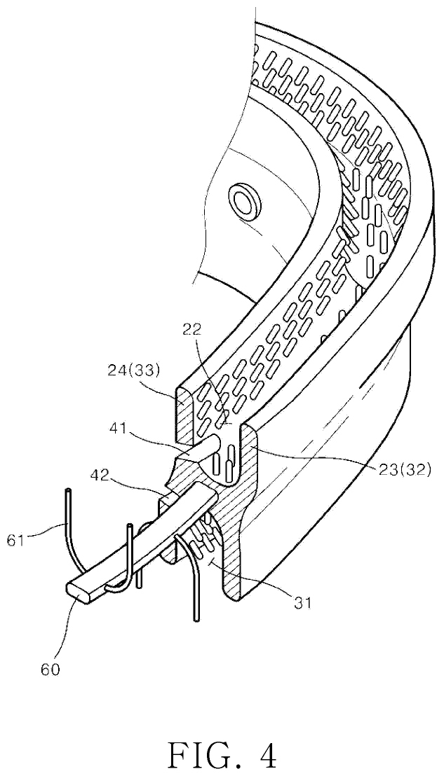

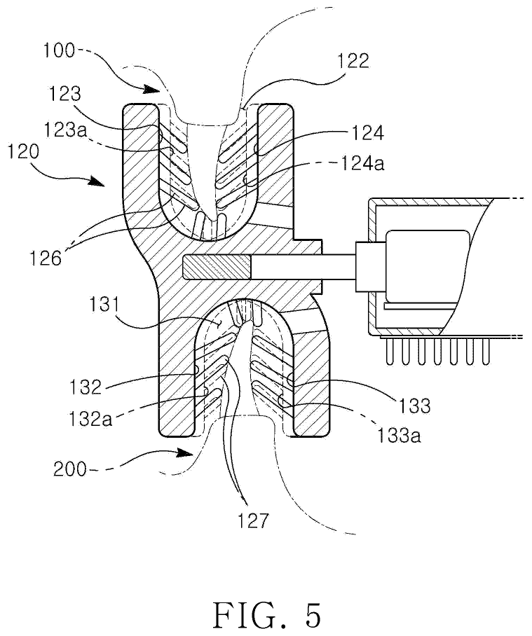

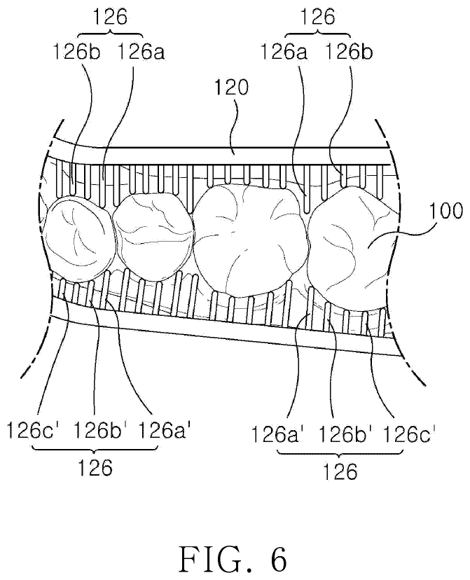

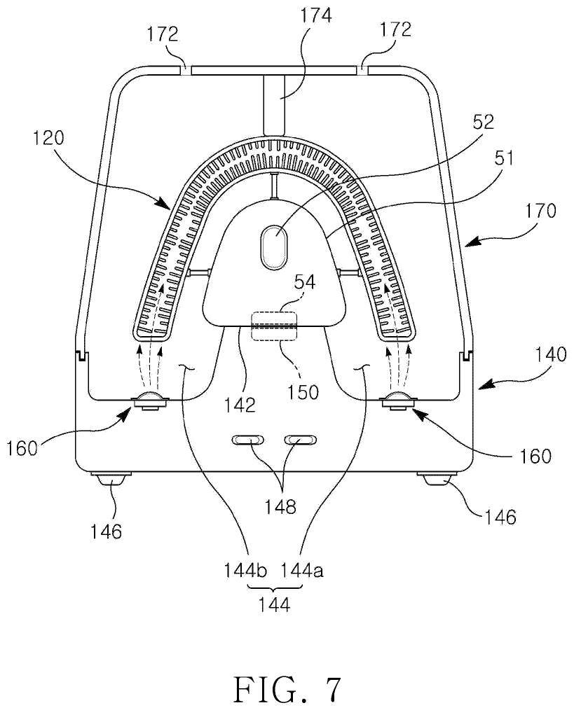

FIG. 1 is a perspective view of a mouthpiece-type electric toothbrush according to the present disclosure, FIG. 2 is a cross-sectional view of the mouthpiece-type electric toothbrush shown in FIG. 1, FIG. 3 is an exploded perspective view showing a toothbrush body and an actuator body of the mouthpiece-type electric toothbrush according to the present disclosure, FIG. 4 is a perspective view showing a portion of the mouthpiece-type electric toothbrush, FIG. 5 is a plan view showing the mouthpiece-type electric toothbrush that has been formed to fit a tooth mold of teeth, FIG. 6 is a cross-sectional view showing a mouthpiece-type electric toothbrush according to an embodiment of the present disclosure, and FIG. 7 is a projective front view showing the state in which a mouthpiece-type electric toothbrush has been mounted on a cleaning device.

A preferable mouthpiece-type electric toothbrush and a cleaning device for the mouthpiece-type electric toothbrush may be modified by those skilled in the art and are a mouthpiece-type electric toothbrush and a cleaning device for the mouthpiece-type electric toothbrush in an embodiment of the present disclosure.

FIG. 1 is a perspective view of a mouthpiece-type electric toothbrush according to the present disclosure, FIG. 2 is a cross-sectional view of the mouthpiece-type electric toothbrush shown in FIG. 1, FIG. 3 is an exploded perspective view showing a toothbrush body and an actuator body of the mouthpiece-type electric toothbrush according to the present disclosure, and FIG. 4 is a perspective view showing a portion of the mouthpiece-type electric toothbrush.

A mouthpiece-type electric toothbrush according to the present disclosure is described with reference to FIGS. 1 to 4. An electric toothbrush 10 for oral insertion according to the present disclosure has a shape corresponding to the arrangement direction of teeth. For example, the electric toothbrush 10 for oral insertion includes: a toothbrush body 20 formed in a horseshoe shape and configured to be inserted in a mouth to be positioned between the upper teeth and the lower teeth when cleaning the teeth; and an ultrasonic wave generator 50 coupled to the toothbrush body 20 to generate ultrasonic vibration.

The toothbrush body 20 is formed in a horseshoe shape and can be inserted into a mouth. The toothbrush body 20 may be made of silicone that is flexible synthetic resin. However, the toothbrush body 20 is not limited thereto. Silver nanopowder may be mixed when forming the toothbrush body 20 using silicone in order to improve the sterilizing power.

The toothbrush body 20 has a base 21 with which the ends of upper and lower teeth can come in contact, as shown in FIGS. 1 and 2. A first tooth insertion groove 22 in which upper teeth are inserted is formed at both sides on the top of the base 21. The first tooth insertion groove 2 has first and second sides 23 and 24.

The first and second sides 23 and 24 may be formed to be placed close to the front surface and the inner surface of upper teeth 100 at a height at which they can massage and clean the ends of upper gums. First bristles 26 and 27 for cleaning the upper teeth 100 are disposed on the inner surfaces of the first and second sides 23 and 24. The first and second bristles 26 and 27 are inclined such that the ends face the upper portion of the first tooth insertion groove 22. For example, the first and second bristles 26 and 27 are inclined to face the ends of the first and second sides 21. First base bristles 28 for cleaning the ends of the upper teeth 100 are disposed on the bottom inside the first tooth insertion groove 22 on the top of the base 21.

A second tooth insertion groove 31 in which lower teeth 200 are inserted is formed at both sides on the bottom of the base 21. The second tooth insertion groove 31 has third and fourth sides 32 and 33. The third and fourth sides 32 and 33 may be formed to be placed close to the front surface and the inner surface of lower teeth 200 at a height at which they can massage and clean the ends of lower gums. First bristles 36 and 37 for cleaning the lower teeth 200 are disposed on the inner surfaces of the third and fourth sides 32 and 33. The third and fourth bristles 36 and 37 are inclined such that the ends face the upper portion of the second tooth insertion groove 31. For example, third and fourth bristles 36 and 37 are inclined to face the upper ends of the third and fourth sides 32 and 33. Second base bristles 38 for cleaning the ends of the lower teeth 200 are disposed on the bottom inside the second tooth insertion groove 31 on the bottom of the base 21. The first tooth insertion groove 22 and the second tooth insertion groove 31 are biased with respect to each other, and the second tooth insertion groove 31 is positioned inside further than the first tooth insertion groove 22. For example, first tooth insertion groove 22 and the second tooth insertion groove 31 may be fitted on the actuator body. The present disclosure is not limited to this configuration and may be modified in accordance with the row of teeth of a user, and the ends of the first, second, third, and fourth bristles and the ends of the first and second base bristles may be rounded to protect gums.

First and second cleaning fluid flow grooves 41 and 45 that a cleaning fluid or bubbles flow into or out of are formed through the second side 24 and the fourth side 33. The first and second cleaning fluid flow grooves 41 and 45 prevent a cleaning fluid and bubbles from overflowing the first and second tooth insertion grooves 22 and 31 when the upper teeth 100 and the lower teeth 200 are cleaned.

The ultrasonic vibration generator 50, which vibrates the toothbrush body 20 to simultaneously clean the upper teeth 100 and the lower teeth 200, is disposed inside the horseshoe-shaped toothbrush body 20, that is, the curved portion.

A circuit board, ultrasonic motors for driving the toothbrush body 20, and a charger (secondary battery) for driving the ultrasonic motors are disposed inside the actuator body 51 of the ultrasonic vibration generator 50. For example, a first ultrasonic vibration motor 55 is disposed close to the front inside the actuator body 51, and second and third ultrasonic vibration motors 56 and 57 are disposed close to both sides inside the actuator body 51, respectively. The inner surface of the actuator body 51 may be coated with a thin metal layer for blocking electromagnetic waves.

First, second, and third driving shafts 55a, 56a, and 57a of the first, second, and third ultrasonic vibration motors 55, 56, and 57 are coupled to the toothbrush body 20 to vibrate the toothbrush body 20. To this end, first, second, and third driving shaft couplers 55b, 56b, and 57b are formed on the toothbrush body 20. In order to easily couple and separate the first, second, and third driving shafts 55a, 56a, and 57a and the first, second, and third driving shaft couplers 55b, 56b, and 57b to and from each other, the second and third driving shaft couplers 56b and 57b may be inclined at a predetermined angle toward the toothbrush body 20, as shown in FIG. 1.

A vibration accelerator 60 is disposed in the base 21 to accelerate vibration by the first, second, and third driving shafts 55a, 56a, and 57a. The vibration accelerator 60 disposed along and inside the base 21 may be a strip-shaped hard plastic or a flat spring. The vibration accelerator 60, as shown in FIG. 4, includes assistant vibration accelerators 61 extending inside the first, second, third, and fourth sides 23, 24, 33, and 34 from the vibration accelerator 60.

The assistant vibration accelerators 61 may have a cross-sectional area smaller than that of the vibration accelerator 60 so that vibration can be transmitted well.

The actuator body 51 further has a tongue cleaner 70 for clean a tongue on the bottom thereof. The tongue cleaner 70 includes a brush 71 for cleaning a tongue on the bottom of the actuator body 51 by vibration of the actuator body 51. The brush 71 has cleaning bristles 72 for cleaning. Ends of the cleaning bristles 72 may be rounded not to damage the tongue when cleaning the tongue. The brush 71 disposed on the bottom of the actuator body 51 may further include a part for coupling and separating to and from the bottom of the actuator body 51.

A control circuit board 300 for driving the first, second, and third ultrasonic vibration motors 55, 56, and 57, and a battery for supplying power may be installed in the actuator body 51. A switch for driving the first, second, and third ultrasonic vibration motors 55, 56, and 57 may be installed at the actuator body 50, but the present disclosure is not limited thereto and a remote controller for remote control may be further provided.

Cleaning blades 81, 82, 83, and 84 for cleaning the inner sides of back teeth at the ends of a row of teeth may be formed at the ends of the first and second sides 23 and 24 and the ends of the third and fourth sides 32 and 33. The cleaning blades 81, 82, 83, and 84 have a structure of which the end extends to be able to surround the rearmost back teeth, as shown in FIG. 4.

Meanwhile, the mouthpiece-type electric toothbrush may further include a charger for charging the secondary battery in the actuator body 51. The charger may have a UV lamp for sterilizing the toothbrush body 20 using UV light when charging the mouthpiece-type electric toothbrush. The secondary battery may be wirelessly charged by the charger.

In order to clean teeth, that is, the upper teeth 100 and the lower teeth 200 using the mouthpiece-type electric toothbrush having the above configuration according to the present disclosure, a user injects a cleaning fluid into the first and second tooth insertion grooves 22 and 31 of the toothbrush body 20, opens his/her mouth, inserts the upper teeth 100 and the lower teeth 200 into the first and second tooth insertion grooves 22, respectively, and then closes the mouth. In this case, the lips are closed. Further, the brush 71 on the bottom of the toothbrush body 20 is brought in contact with the user's tongue. The cleaning fluid may be liquid toothpaste.

The first, second, and third ultrasonic vibration motors 55, 56, and 57 disposed in the actuator body 50 are driven. Accordingly, the vibration generated by the first, second, and third ultrasonic vibration motors 55, 56, and 57 is transmitted to the toothbrush body 20, and the upper teeth 100 are cleaned by the first and second bristles 26 and 27 disposed on the first and second sides 23 and 24 of the toothbrush body 20 and the first base bristles 28. Further, the lower teeth 200 are cleaned by the third and fourth bristles 36 and 37 on the third and fourth sides 32 and 33 and the second base bristles 88, and the top of the tongue is cleaned by the brush 71 of the tongue cleaner 70 on the bottom of the actuator body 51.

Since the first, second, and third driving shafts 55a, 56a, and 57a of the first, second, and third ultrasonic vibration motors 55, 56, and 57 are coupled to or in contact with the vibration accelerator 60 while the upper teeth 100 and the lower teeth 200 are cleaned, as described above, the ultrasonic vibration generated by the first, second, and third ultrasonic vibration motors 55, 56, and 57 is transmitted well to the toothbrush body 20. Further, the cleaning fluid and bubbles move to the inside of the mouth, that is, toward the vibration body 50 through the first and second cleaning fluid flow grooves 41 and 45 formed in the second and third sides 23 and 33, respectively, while the teeth are cleaned. When the cleaning fluid is insufficient in the first tooth insertion groove 22 in which the upper teeth 100 are inserted and in the second tooth insertion groove 32 in which the lower teeth 200 are inserted, the cleaning fluid and bubbles in the mouth are supplied through the first and second cleaning fluid flow grooves 41 and 45, whereby the teeth are cleaned well.

According to the electric toothbrush 10 for oral insertion of the present disclosure, as described above, since upper teeth and lower teeth are cleaned with the electric toothbrush full inserted in a mouth and lips closed, it is possible to prevent the cleaning fluid from flowing out of the mouth. Further, since teeth are cleaned with the electric toothbrush fully inserted in a mouth, the user can feely use both hand while the teeth are cleaned. In particular, the disabled or the elderly and weak can be less helped by assistants when cleaning teeth.

FIG. 5 is a plan view showing the mouthpiece-type electric toothbrush that has been formed to fit a tooth mold of teeth and FIG. 6 is a cross-sectional view showing a mouthpiece-type electric toothbrush according to an embodiment of the present disclosure.

A mouthpiece-type electric toothbrush according to the present disclosure is described with reference to FIGS. 5 and 6. A mouthpiece-type electric toothbrush includes a toothbrush body 120 having a first tooth insertion groove 122 in which the upper teeth of a user are inserted and a second tooth insertion groove 131 in which the user's lower teeth are inserted.

The toothbrush body 120 has bristles 126 and 127 having different lengths and formed in the first tooth insertion groove 122 and the second tooth insertion groove 131 in accordance with a tooth mold (not shown) patterned after the row of teeth of a user. Patterning the row of teeth of a user means various methods such as forming a mold by patterning the row of teeth of a user using a soft material and then injecting plaster, and printing a mold by scanning and printing teeth using a 3D printer, and tooth molds can be obtained by these methods. The tooth mold is inserted in a molding (not shown) and then the toothbrush body 120 is formed such that bristles protrude toward teeth.

The toothbrush body 120 may be formed by inserting a thermoplastic material to fit the row of teeth of a user at a predetermined temperature. For example, a tooth mold is formed first to form the toothbrush body 120. The tooth mold is formed by pattering in the same manufacturing method as the way that scuba divers make personal mouthpieces and then bristles can be formed like the toothbrush 120 of the present disclosure. The method of manufacturing mouthpieces is generally known in the art, so it is not described in detail. The tooth mold may be formed by scanning and printing the row of teeth using a 3D printer other than using the method of patterning teeth.

First bristles 123 are arranged with predetermined gaps therebetween along the inner surfaces of a first side 123, 123a and a second side 124, 124a of the first tooth insertion groove 122. The first bristles 123 are formed in the toothbrush 120 formed from the tooth mold patterned after the row of teeth of a user. The first bristles 126 may have different lengths and shapes to fit the row of teeth of a user.

For example, the first bristles 126 are formed to face each other on the first side 123, 123a and the second side 124, 124a to fit to the length, thickness, etc. of the teeth of a user. The first bristles 126 have different lengths so that the first bristles 126 on the first side 123, 123a and the first bristle 126 on the second side 124, 124a can come in contact with teeth, respectively. The first bristles 126 are in contact with teeth, respectively, and inserted between the teeth, thereby being able to remove foreign matters such as plaque. For example, the bristles 126a′, 126b′, and 126c′ extending from the first side 123 and the bristles 126a and 126b extending from the second side 124 of the first bristles 126 have different lengths, which are set such that the bristles can be inserted between teeth.

Second bristles 127 are arranged with predetermined gaps therebetween along the inner surfaces of a third side 132, 132a and a fourth side 133, 133a of the second tooth insertion groove 131. The second bristles 127 are formed in the toothbrush 120 formed from the tooth mold patterned after the row of teeth of a user. The second bristles 127 may have different lengths and shapes to fit the row of teeth of a user.

For example, the second bristles 127 are formed to face each other on the third side 132, 122a and the fourth side 133, 133a to fit to the length, thickness, etc. of the teeth of a user. The first bristles 127 have different lengths so that the first bristles 126 on the first side 123, 123a and the first bristle 127 on the second side 124, 124a can come in contact with teeth, respectively. The second bristles 127 are in contact with teeth and inserted between the teeth, respectively, thereby being able to remove foreign matters such as plaque.

FIG. 7 is a projective front view showing the state in which a mouthpiece-type electric toothbrush according to the present disclosure has been mounted on a cleaning device.

A mouthpiece-type electric toothbrush according to the present disclosure is described with reference to FIG. 7. The mouthpiece-type electric toothbrush includes: a toothbrush body 120 having a first tooth insertion groove 122 in which the upper teeth of a user are inserted and a second tooth insertion groove 131 in which the lower teeth of a user are inserted; and a body cradle 140 on which an actuator body 51, which has an ultrasonic wave generator (50 in FIG. 3) mounted on the toothbrush body 120 and transmitting vibration to the first tooth insertion groove 122 and the second tooth insertion groove 131, is placed, and that washes and/or dries the first tooth insertion groove 1222 and the second tooth insertion groove 131.

The body cradle 140 has: a body seat 142 on which the actuator body 51 of the electric toothbrush 10 is seated; recessed grooves 144 formed at both sides on the body seat 152; and sterilizing modules 160 dry and/or sterilize the electric toothbrush 10 on the recessed grooves 144. The body cradle 140 includes a cover 170 coupled to the body cradle 140 to protect the electric toothbrush 10 and prevent UV light for sterilization from leaking outside. The body cradle 140 has the body seat 142 on which the actuator body is seated, and further includes a charging module 15 disposed on the body seat 142.

The body cradle 140 may have a display 148 to be able to inform a user of the state of the charging module 150 and/or whether the sterilizing modules 160 are operated. The display 148 can be shown as red when the toothbrush body is being charged and as green when it is fully charged, in order to show the state of the charging module 15 to a user. The display colors are not limited to red and green, and it is possible to show various colors to a user such as red, blue, green, by controlling an LED.

The charging module 150 charges the toothbrush body 120. The charging module 150 may use a wireless charging method and a wired charging method to charge the toothbrush body 120, but uses a wireless charging method as in the above embodiment of the present disclosure. When the toothbrush body 120 is placed on the charging module 150, the toothbrush body 120 is charged even without an additional cable, so the toothbrush body 120 can be charged anytime anywhere. The charging module 150 and/or the toothbrush body 120 may be equipped with an anti-overcharging device to prevent overcharging of a battery.

The sterilizing modules 160 dry and/or sterilize the toothbrush body 51 on the recessed grooves 144. The sterilizing module 160 may be at least any one of a UV lamp and a blowing fan, but a UV lamp is exemplified in an embodiment of the present disclosure. UV lamps are developed to output high efficiency and high power as the application range and the use scale are increased. Further, the lifespan of the lamps are being increased by development of a ballast for turning on/off the lamps. Accordingly, the lamps and the ballast have a complementary relationship and are recently developed in a variable power type that can control power within an appropriate range, so the amount of UV light that is emitted by the lamps can be maintained at a predetermined level. The sterilization effect by UV light is obtained in a UVC range between 200 nm and 280 nm of the UV wavelength band.

The wavelength showing the largest sterilization effect is 253˜270 nm. The wavelength of 254 nm that is emitted by low-voltage or high-power low-voltage amalgam lamps also has a very large UV sterilization effect, but the lamps emit only 254 nm for the characteristics thereof. Accordingly, although it is known that the wavelength shows the largest effect, the largest effect is actually obtained at 265 nm.

UV lamps having the same type as the standard of common fluorescent lamps as a kind of UV lamps has too low efficiency and too large volume to be used for industry, so they are used for home, individuals, or tests. A low-voltage/low-power lamp is a lamp that has been generally used for industry. Such a lamp is a lamp having a small diameter, high efficiency, and one connection wire for each of both sides, as compared with fluorescent lamps (fluorescent lamps are bidirectional 2-wire type lamps). Since the specifications are standards, popular makers produce most of the lamps in the world. Further, since the prominent characteristic of the low-voltage/low-power lamps is the standardized universality, they are usually called standard lamps. Further, since the efficiency is high and the UV density per unit cm is 4˜5 times higher as compared with low-voltage/low-output lamps, it is possible to deal with a large capacity. They have a characteristic of a multiwave and have a multiwave from 185 nm to about 600 nm. Since the UV density is tens of times larger than a low-voltage lamp, they have a short response time is short and are suitable for narrow places. Further, since energy is concentrated, the surface temperature of the lamps is 600˜800° C.

Meanwhile, UV light is classified into UV-A: 320˜0 nm, UV-B: 280˜320 nm, UV-C: 100˜280 nm in accordance with the wavelength. As for production of ozone (184.9 nm), when UV light is emitted to the oxygen in the air, some of the oxygen molecules are change into ozone, thereby generating a sterilizing and deodorizing effect. Further, as for sterilization (253.7 nm), UV light has an excellent sterilization effect in the air and water, so it can be used for various fields. As for erythema (297 nm), it is a phenomenon in which the skin is exposed to UV light, the skin becomes rough and a pain is accompanied. A photochemistry act (365 nm) is a chemical reaction that is generated by light, and can be applied to the actual life in various ways through a hardening principle. As for production of negative ions (150˜200 nm), harmful positive ions are neutralized, so it has a function of purifying air and removing contaminants.

The reason that UV-C is specified as sterilizing UV light is that the DNA in cells absorbs well the UV-C. Further, since the wavelength of 253.7 nm (2537) is most well absorbed in DNA, so UV lamps can most effectively emit the wavelength of 253.7 nm. Accordingly, the UV light of a double wavelength of 253.7 nm has very strong sterilization power, so it is also called sterilizing light and has sterilization power about 1,000˜10,000 times higher than UV-A and UV-B that have relatively large wavelengths. Accordingly, the UV light keels colitis germs and common bacteria within 6˜10 seconds.

However, it is difficult to sense UV-C wavelength at places with clean air such as the sea or mountains, and the reason is that the UV-C wavelength has low permeation power because it is too short and the ozone layer existing in the atmosphere of the earth completely blocks the UV-C. Sterilization by UV light is a photooxidation effect for DNA, and the term ‘inactivation’ is used as a terminology. In detail, when the enzyme in DNA receives light, damaged DNA is recovered and microorganisms are activated, which is called photorecovery. When the sterilizing module 160 is operated, a user can know the situation from the light emitted from the sterilizing module 160, but it is possible to inform a user of whether the electric toothbrush is operated, by providing the display 148 to the body cradle 140.

The cover 170 can block UV light that is emitted by a light source, which emits light when the sterilizing modules 160 are operated, or that is emitted by the sterilizing modules 160, or can prevent water from flying from the toothbrush body 120 and/or the toothbrush body 120 from being contaminated due to exposure to the outside. The cover 170 may be provided by coating to block the UV light that is emitted from the UV lamps of the sterilizing modules 160.

An outlet 172 may be formed at the upper portion of the cover 170 so that vapor can be easily discharged when the toothbrush is sterilized and dried. The cover 170 is fixed to the lower charging part and the upper fixing part of the toothbrush under predetermined pressure by a rotary type lock that is locked at a predetermined angle, thereby providing an effect of preventing the toothbrush body from shaking when it is carried.

In general, when a toothbrush is placed on a charger and then the cover 170 is closed, charging and sterilizing are automatically performed.

Since teeth are cleaned with the electric toothbrush inserted in a mouth, as described above, it is possible to prevent the cleaning fluid for teeth and bubbles from flowing or flying out of the mouth. Further, since the first and second discharge guide grooves are formed in the second and fourth sides, respectively, the cleaning fluid and bubbles are smoothly discharged from the first and second tooth insertion grooves and smoothly flow into the first and second tooth insertion grooves from the oral cavity. Further, since the vibration generated by the first, second, and third ultrasonic vibration motors can be transmitted well to the parts of the toothbrush body by the vibration accelerator disposed in the toothbrush body, the power for cleaning teeth can be increased.

Further, since a user can freely use both hands while the teeth are cleaned, it is possible to reduce the inconvenience that the user has to brush his/her teeth when he/she is busy, and the disabled and the elderly and weak who have difficulty in moving both hands can easily brush their teeth without help by an assistant. Further, it is possible to expect the effect of not only cleaning teeth, but massaging the gums and it is possible to remove plaque and tartar using ultrasonic vibration. Further, a delay time of several seconds is provided until the actuator body is operated after liquid toothpaste is applied to the toothbrush body and an operation switch is turned on before the toothbrush is put into a mouth, whereby it is possible to prevent the toothpaste from flying outside the mouth.

Further, the secondary battery for driving the ultrasonic vibration generator of the actuator body can be wirelessly charged and the actuator body has a shield for blocking electromagnetic waves, so it is possible to fundamentally prevent problems due to electromagnetic waves in a mouth. Further, since even though a user has a set of non-uniform teeth, the bristles uniformly come in contact with the teeth and uniformly clean the teeth. Further, since the mouthpiece-type electric toothbrush is pasteurized and sterilized for a predetermined time through the body cradle on which the mouthpiece-type electric toothbrush is placed, the mouthpiece-type electric toothbrush can be always provided in a clean state. Further, when the mouthpiece-type electric toothbrush is placed on the body cradle, it can be wirelessly charged, so there is an effect that convenience is improved.

The features, structures, effects, etc. exemplified in each embodiment may be combined or modified also in other embodiments by those skilled in the art to which the embodiment are pertained. Accordingly, configurations related to the combinations and modifications should be construed as being included in the range of the present disclosure.

### DESCRIPTION OF REFERENCE NUMERALS

10: mouthpiece-type electric toothbrush
20: toothbrush body

1. A mouthpiece-type electric toothbrush, comprising:
a base formed to have a shape corresponding to the arrangement direction of teeth;
a toothbrush body having first and second sides that are formed at an upper portion of the toothbrush body, form a first tooth insertion groove in which upper teeth are inserted, and have first bristles on inner surfaces thereof, and third and fourth sides that are formed at a lower portion of the base, form a second tooth insertion groove in which lower teeth are inserted, and have second bristles on inner surfaces thereof;
an actuator body disposed inside the toothbrush body at a portion corresponding to the tongue in a mouth; and
an ultrasonic vibration generator disposed in the actuator body and including at least one vibration motor of which a driving shaft is inserted in the toothbrush body.

2. The mouthpiece-type electric toothbrush of claim 1, wherein the ultrasonic vibration generator includes: a first vibration accelerator disposed in the base of the toothbrush body; first and second ultrasonic vibration motors disposed at both sides in the actuator body and having first and second driving shafts connected to the vibration accelerator; and a third ultrasonic vibration motor disposed at a front inside the actuator body and having a third driving shaft connected to the vibration accelerator disposed in the base.

3. The mouthpiece-type electric toothbrush of claim 2, wherein the vibration accelerator disposed in the base has assistant vibration accelerators extending from the vibration accelerator and extending in the first, second, third, and fourth sides.

4. The mouthpiece-type electric toothbrush of claim 1, wherein a first discharge guide groove for guiding and discharging bubbles and a cleaning fluid from the first tooth insertion groove toward the portion inside teeth in a mouth is formed at the second side, and
a second discharge guide groove for bubbles and a cleaning fluid from the second tooth insertion groove toward the portion inside teeth in a mouth is formed at the fourth side.

5. The mouthpiece-type electric toothbrush of claim 1, wherein a cleaning blade for cleaning inner surfaces of back teeth at ends of a row of teeth is further formed at an end of each of the first and second sides and an end of each of the third and fourth sides.

6. The mouthpiece-type electric toothbrush of claim 1, wherein vertical centers of the first tooth insertion groove and the second tooth insertion groove are biased from each other.

7. The mouthpiece-type electric toothbrush of claim 2, wherein the driving shafts of the first and second motors are inclined toward an inlet of a mouth to be easily coupled to and separated from the vibration accelerator disposed in the base.

8. A mouthpiece-type electric toothbrush, comprising a toothbrush body having a first tooth insertion groove in which the upper teeth of a user are inserted and a second tooth insertion groove in which the lower teeth of the user are inserted,
wherein the toothbrush body has a plurality of bristles having different lengths and formed in the first tooth insertion groove and the second tooth insertion groove in accordance with a tooth mold patterned after a row of teeth of the user.

9. The mouthpiece-type electric toothbrush of claim 8, wherein the toothbrush body is formed by inserting a thermoplastic material to fit a row of teeth of a user at a predetermined temperature.

10. A mouthpiece-type electric toothbrush, comprising:
a toothbrush body having a first tooth insertion groove in which the upper teeth of a user are inserted and a second tooth insertion groove in which the lower teeth of the user are inserted;
an actuator body having a ultrasonic vibration generator mounted on the toothbrush body and transmitting vibration to the first tooth insertion groove and the second tooth insertion groove; and
a body cradle on which the actuator body is placed and that is configured to wash and/or dry the first tooth insertion groove and the second tooth insertion groove.

11. The mouthpiece-type electric toothbrush of claim 10, wherein the body cradle has a body seat on which the actuator body is seated, and
a charging module configured to charge the actuator body is disposed at the body seat.

12. The mouthpiece-type electric toothbrush of claim 11, wherein the body cradle has recessed grooves respectively formed at both sides with the body seat therebetween, and
a sterilizing module configured to dry and/or sterilize the toothbrush body is disposed at each of the recessed grooves.

13. The mouthpiece-type electric toothbrush of claim 12, wherein the sterilizing module is any one of a UV lamp and/or a blowing fan.

14. The mouthpiece-type electric toothbrush of claim 13, wherein the sterilizing modules are inclined at a predetermined angle toward the first tooth insertion groove and the second tooth insertion groove.

15. The mouthpiece-type electric toothbrush of claim 12, further comprising a cover applied by coating to block UV light that is emitted from the UV lamp.
